(19) United States
(12) Patent Application Publication
(10) Pub. No.: US 2024/0016405 A1
(43) Pub. Date: Jan. 18, 2024

(54) DETECTING AND COLLECTING ANALYTE DATA

(71) Applicant: Know Labs, Inc., Seattle, WA (US)

(72) Inventor: Phillip BOSUA, Seattle, WA (US)

(21) Appl. No.: 17/865,806

(22) Filed: Jul. 15, 2022

(51) Int. Cl. A61B 5/0507; A61B 5/145; A61B 5/00; G16H 50/30

(52) U.S. Cl. CPC A61B 5/0507; A61B 5/14532; A61B 5/14546; A61B 5/7275; G16H 50/30; A61B 5/681

(57) ABSTRACT

Establishing an analyte database using analyte data that has been obtained using one or more non-invasive analyte sensors, and using the analyte database to analyze data obtained using a non-invasive analyte sensor. Once the analyte database is established, the analyte database can be updated with new analyte data, and the analyte database can be used to analyze the new analyte data to derive information from the new analyte data. For example, in the case of a human target, the new analyte data together with the analyte database can be used to predict an actual or possible abnormal medical pathology of the human target.

### FIELD

This technical disclosure relates to apparatuses, systems, programs, and methods of establishing an analyte database using analyte data that has been obtained using one or more non-invasive analyte sensors.

### BACKGROUND

A sensor that uses radio or microwave frequency bands of the electromagnetic spectrum for non-invasive collection of analyte data of a subject is disclosed in U.S. Pat. No. 10,548,503. Additional examples of sensors that purport to be able to use radio or microwave frequency bands of the electromagnetic spectrum to detect an analyte in a person are disclosed in U.S. Patent Application Publication 2019/0008422 and U.S. Patent Application Publication 2020/0187791.

### SUMMARY

This disclosure relates generally to establishing an analyte database using analyte data that has been detected and collected using non-invasive analyte sensor(s). Once the analyte database is established, the analyte database can be cyclically updated with new analyte data, which may then be used for, at least, analysis and/or the detection of trends.

The analyte data used to establish the analyte database is obtained over a period of time from a plurality of human or animal subjects (or collectively subjects), from a plurality of animate or inanimate materials, or from a plurality of other objects. The human or animal subjects, the animate or inanimate materials, and any other objects from which analyte data is obtained using the non-invasive analyte sensors may collectively be referred to as targets. The targets used to establish the analyte database are similar to one another. For example, the targets can be humans; the targets can be the same kind of animal such as cows (or breed of cows); the targets can be the same kind of trees (such as apple trees) or the same kind of fluid such as fuel, oil, hydraulic fluid, edible or potable liquids, or the like. Further, an analyte may be detected from a fluid, for example blood, interstitial fluid, cerebral spinal fluid, lymph fluid or urine; human tissue; animal tissue, plant tissue, an inanimate object, soil, genetic material, or a microbe.

In another embodiment, analyte data used to establish the analyte database is obtained over a period of time from a single target so that the analyte database is specific to a single target. Additional analyte data can then be obtained from the target, with the analyte database being updated with the additional analyte data.

The term “analyte” used herein refers to a substance for which constituents are being identified and/or measured. For example, glucose is a sugar that is a component of many carbohydrates. The analyte is present in a host which can be a liquid, gas, solid, gel, and combinations thereof.

The analyte data stored in the analyte database may be raw, unprocessed data that is obtained by the analyte sensor. The raw, unprocessed data may then be analyzed to extract out data regarding the analyte such as the physical presence of the analyte in a corresponding host and/or a volume or concentration of the analyte in the host or target. The analyte data stored in the analyte database may alternatively be previously processed data regarding the analyte such as the physical presence of the analyte in the host or target and/or a volume or concentration of the analyte in the host or target. The analyte data stored in the database may also be a combination of raw, unprocessed data and processed data. Regardless of the form of the analyte data stored in the analyte database, the analyte data contains information regarding at least one analyte in the targets. In an example by which the targets are human or animal subjects, the analyte may be an indicator of an abnormal (or normal) medical pathology of the subjects. In an example where the targets are animate or inanimate materials, the analyte may be an indicator of an abnormal (or normal) condition of the materials such as, but not limited to, a contaminant or other impurity in the materials, a disease condition of the materials, a mineral in soil, and many others.

The analyte data used to establish the analyte database is collected over a period of time that is sufficient to eliminate or minimize the effects of temporary variations or aberrations in the analyte of the targets. This helps to ensure that an accurate actual or possible abnormal (or normal) indicator in the subsequently obtained analyte data can be determined based on the analyte database. The time period may vary based on a number of factors including, but not limited to, the target, the analyte being detected, temporal factors (for example time of day, the day(s) of the week, month or year), and other factors.

The time period over which the analyte data is collected can be measured over a range of time that may be measured in seconds, minutes, hours, days, months or even years. In one embodiment, the time period can be selected to minimize or avoid collecting analyte data encompassing natural or non-abnormal variations in the analyte of the target that may occur and that may not indicate an actual or possible abnormal condition. In another embodiment, the time period that is selected may include collecting analyte data that encompasses natural or normal variations in the analyte of the target that may occur whether or not the collected analyte data indicates an actual or possible abnormal condition.

The analyte data is collected using non-invasive analyte sensors that detect an analyte in the target via spectroscopic techniques using non-optical frequencies such as in the radio or microwave frequency range of the electromagnetic spectrum or optical frequencies in the visible range of the electromagnetic spectrum. In one embodiment, the analyte sensors described herein can be used for in vivo detection of the analyte data or used for in vitro detection of the analyte data from the target.

In one embodiment, data may also be collected from the target using a second sensor from which the data from the second sensor together with the analyte data collected by the analyte sensor, can be used to predict an actual or possible abnormal (or normal) condition of the target.

In one embodiment, the techniques described herein can be used on human or animal subjects for determining an abnormal (or alternatively a normal) medical pathology. For example, in one embodiment, a method described herein can include establishing an analyte database that is based on analyte data that has been obtained from subjects using non-invasive analyte sensors that have each conducted a plurality of analyte sensing routines on the subjects to obtain the analyte data from the subjects over a period of time including, but not limited to, at least twenty-four hours. The analyte data may contain information regarding at least one analyte in the subjects, with the at least one analyte being an indicator of an abnormal medical pathology. Each non-invasive analyte sensor includes a detector array having at least one transmit element and at least one receive element. For each sensing routine of the plurality of sensing routines, the at least one transmit element is positioned and arranged to transmit an electromagnetic transmit signal into the corresponding subject, and the at least one receive element is positioned and arranged to detect a response resulting from transmission of the electromagnetic transmit signal by the at least one transmit element into the corresponding subject. A transmit circuit is electrically connectable to the at least one transmit element. The transmit circuit is configured to generate the electromagnetic transmit signal to be transmitted by the at least one transmit element, and the electromagnetic transmit signal is in a radio frequency or visible range of the electromagnetic spectrum, as well as a harmonic thereof. In addition, a receive circuit is electrically connectable to the at least one receive element, with the receive circuit being configured to receive the response detected by the at least one receive element.

Once the analyte database is established, new analyte data can be obtained from a subject by a non-invasive analyte sensor. The analyte database can be updated based on the new analyte data, and the new analyte data can be analyzed based on the analyte database.

In another embodiment, analyte data obtained over a period of time from a single target using one or more of the analyte sensors described herein can be used to establish an analyte database whereby the analyte database is specific to a single target. Additional analyte data can then be obtained from the target, the analyte database updated with the additional analyte data.

In another embodiment, an analytics system described herein can include the analyte database and at least one of the non-invasive analyte sensors.

### DRAWINGS

FIG. 1 is a schematic depiction of an analyte sensor system with a non-invasive analyte sensor relative to a target according to an embodiment.

FIGS. 2A-C illustrate different example orientations of antenna arrays that can be used in an embodiment of a sensor system described herein.

FIGS. 3A-3C illustrate different examples of transmit and receive antennas with different geometries.

FIGS. 4A, 4B, 4C and 4D illustrate additional examples of different shapes that the ends of the transmit and receive antennas can have.

FIG. 5 illustrates another example of an antenna array that can be used.

FIG. 6 illustrates another embodiment of an analyte sensor system with a non-invasive analyte sensor according to an embodiment.

FIG. 7 illustrates another embodiment of an analyte sensor system with a non-invasive analyte sensor according to an embodiment.

FIG. 8 illustrates another embodiment of an analyte sensor system with a non-invasive analyte sensor relative to a target according to an embodiment.

FIG. 9 illustrates another embodiment of an analyte sensor system with a non-invasive analyte sensor relative to a target according to an embodiment.

FIG. 10 is a flowchart of a method for detecting an analyte according to an embodiment.

FIG. 11 is a flowchart of analysis of a response according to an embodiment.

FIG. 12 is a schematic depiction of predictive medical analytics system described herein.

FIG. 13 is a schematic depiction of a method of establishing an analyte database and predicting a condition of a target described herein.

FIG. 14 is a schematic depiction of a method of establishing an analyte database using analyte data from a single target.

Like reference numbers represent like parts throughout.

### DETAILED DESCRIPTION

The following is a detailed description of using analyte data that has been collected from targets (or from a single target) by analyte sensors, for example non-invasive analyte sensors, to establish an analyte database and using the analyte database to analyze data obtained from a target using an analyte sensor, for example a non-invasive analyte sensor. Once the analyte database is established, the analyte database can be updated with new analyte data that is collected, and the analyte database can be used to analyze the new analyte data to derive information from the new analyte data. The information can be used to predict or derive an actual or possible condition (abnormal or normal) of the target.

The analyte data stored in the analyte database may be raw, unprocessed data that is obtained by the analyte sensor(s). Raw unprocessed data is data that is obtained by the analyte sensor(s) and that is not processed by the analyte sensor(s) and that does not undergo any other processing prior to being stored in the analyte database. The raw, unprocessed data may then be analyzed to extract out data on the analyte such as the presence of the analyte and/or a concentration of the analyte. The analyte data stored in the analyte database may alternatively be processed data regarding the analyte such as the presence of the analyte and/or a concentration of the analyte, where the processed data results from processing of raw unprocessed data by the analyte sensor(s) and/or by another device prior to being stored in the analyte database. The analyte data stored in the database may also be a combination of raw, unprocessed data and processed data.

The analyte data used to establish the analyte database is obtained over a period of time from a plurality of targets or from a single target. The targets can be human or animal subjects (or collectively subjects), a plurality of animate or inanimate materials, or a plurality of other objects; or, further, cells or tissues thereof. The targets used to establish the analyte database are similar to one another. For example, the targets can be humans; the targets can be the same kind of animal such as dogs (or breed of dogs); the targets can the same kind of trees (such as apple trees) or the same kind of fluid such as fuel, oil, hydraulic fluid, edible or potable liquids, or the like.

The analyte data that is collected contains information on at least one analyte in the targets. In an example where the targets are human or animal subjects, the analyte may be an indicator of an abnormal (or normal) medical pathology of the subjects. In an example where the targets are animate or inanimate materials, the analyte may be an indicator of an abnormal (or normal) condition of the materials such as, but not limited to, a contaminant or other impurity in the materials, a disease condition of the materials, a mineral in soil, and many others conditions.

The analyte data, both for establishing the analyte database and subsequent analyte data for updating the database and for analyzing, may be collected using non-invasive analyte sensors that detect an analyte in the targets via spectroscopic techniques using non-optical frequencies such as in the radio or microwave frequency range of the electromagnetic spectrum or optical frequencies in the visible range of the electromagnetic spectrum. The analyte sensors described herein can be used for in vivo detection of the analyte and in vitro detection of the analyte.

One or more analytes can be detected. The analyte(s) that is detected is an indicator of a condition (abnormal or normal) of the target. For example, when the target is a human, the analyte can be an indicator of an abnormal medical pathology of the human target. For example, the analyte can include, but is not limited to, one or more of glucose, ketones, C-reactive proteins, alcohol, white blood cells, luteinizing hormone or any other analyte that is an indicator of an actual or possible abnormal medical pathology of the human target. The abnormal medical pathology can include, but is not limited to, pre-diabetes, diabetes, cancer, cirrhosis and other medical pathologies that can be predicted based on one or more detectable analytes from the human target.

The time period over which the analyte data (both for establishing the analyte database and subsequent analyte data collection) is collected may vary based on a number of factors including, but not limited to, the target, the analyte being detected, temporal factors (for example time of day, the day(s) of the week, month or year), and other factors. The time period over which the analyte data is collected can be measured in hours, days, months or even years. In one embodiment, the time period can be selected to minimize or avoid collecting analyte data encompassing natural or non-abnormal variations in the analyte of the target(s) that may occur and that may not indicate an actual or possible abnormal (or normal) condition of the target. In another embodiment, the time period that is selected may include collecting analyte data that encompasses natural or normal variations in the analyte of the targets that may occur that may not indicate an actual or possible abnormal condition.

In one embodiment, data may also be collected from the target(s) using a second sensor where the data from the second sensor, together with the analyte data collected by the analyte sensor(s), can be used to predict an actual or possible condition of the target. In another embodiment, analyte data may also be collected from one or more additional targets and the collected analyte data of each target may be used to predict an actual or possible condition of the respective target.

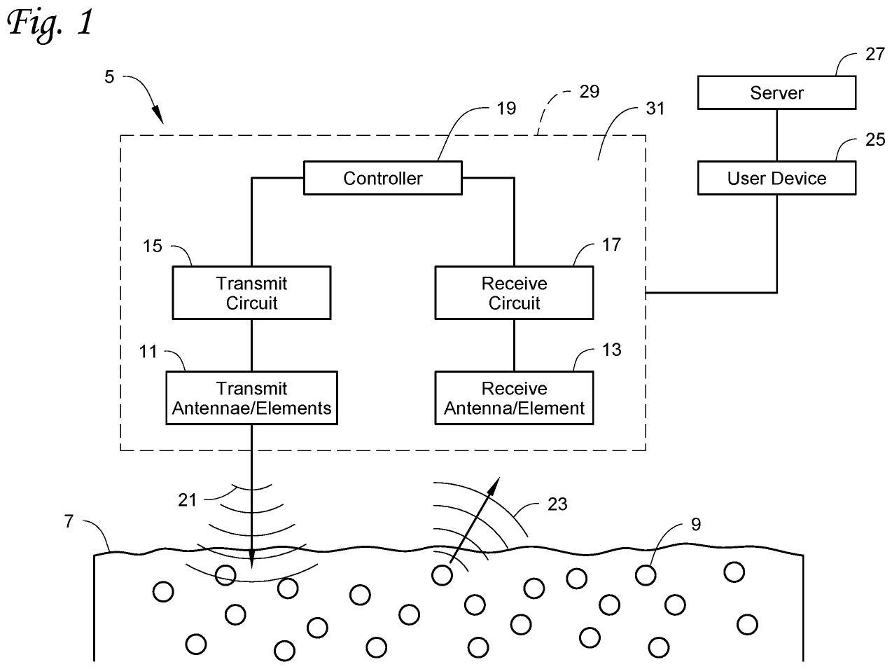

The analyte(s) may be detected via spectroscopic techniques using non-optical frequencies such as in the radio or microwave frequency bands of the electromagnetic spectrum or optical frequencies in the visible range of the electromagnetic spectrum. An analyte sensor described herein includes a detector array having at least one transmit element and at least one receive element. The transmit element and the receive element can be antennas (FIGS. 1-5). In the following description, the transmit element and the receive element, whether they are antennas or light emitting diodes, may each be referred to as a detector element.

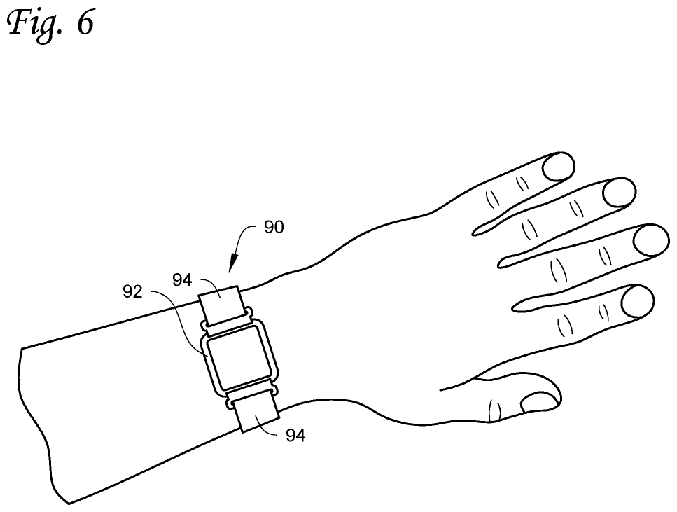

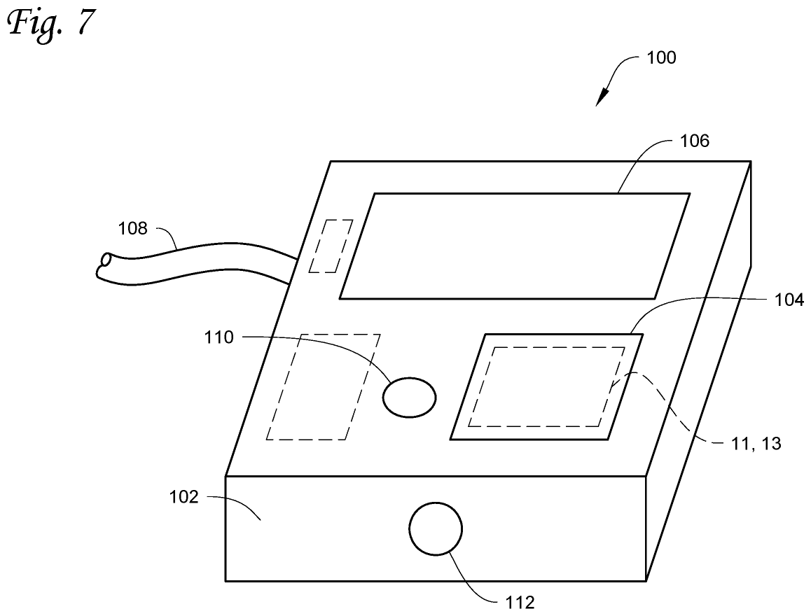

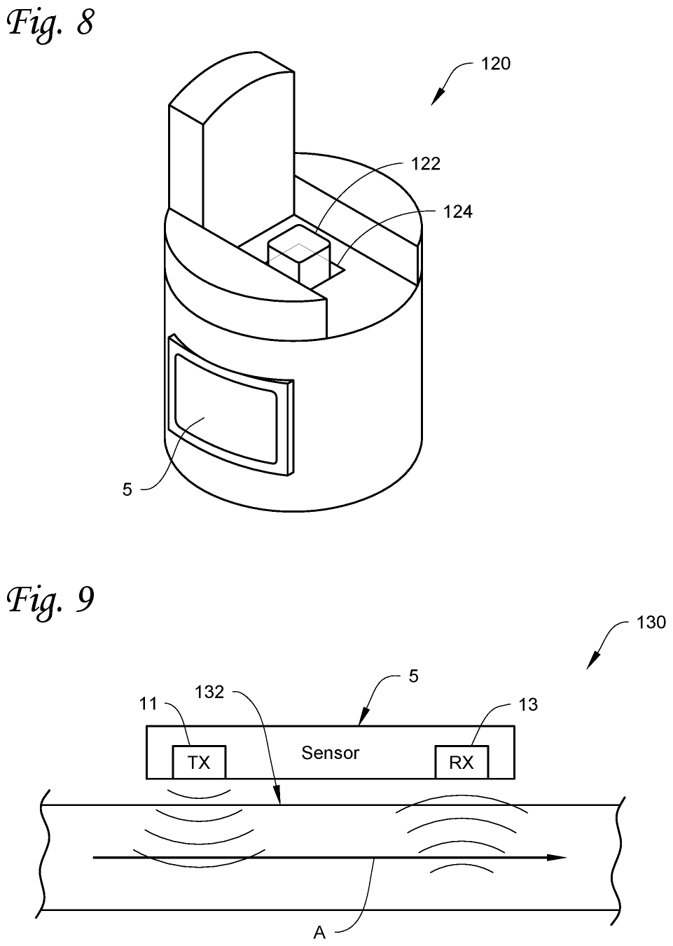

The following description together with FIGS. 1-5 will initially describe the analyte sensor system as including a detector array having two or more antennas. FIG. 6 illustrates an analyte sensor system with a non-invasive analyte sensor in the form of a body wearable sensor, for example worn around the wrist. FIG. 7 illustrates an analyte sensor system with a non-invasive analyte sensor in the form of a tabletop device. FIG. 8 illustrates an analyte sensor system with a non-invasive analyte sensor in the form of an in vitro sensor used with in vitro targets. FIG. 9 illustrates an analyte sensor system with a non-invasive analyte sensor that can be used with industrial processes.

In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not intended to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

For sake of convenience, the following description may describe the target(s) as being a human or animal subject, though alternatively the target(s) may be a cell or tissue of the aforementioned subjects, and the condition of the subject as being an abnormal medical pathology of the subject. However, the targets are not limited to human or animal subjects, and the condition is not limited to abnormal medical pathologies. The targets can be any objects from which one or more analytes can be detected using the analyte sensors described herein. In addition, the condition that is predicted can be any normal or abnormal condition of an object. Additional examples of conditions can include, but are not limited to, the presence or absence of a contaminant or other impurity in the target which may be a gas, liquid, solid, gel, and combinations thereof; a disease condition or lack of a disease condition of the target; a mineral or lack of mineral in soil; and many others.

In one embodiment, the presence of at least one analyte in a target can be detected. In another embodiment, an amount or a concentration of the at least one analyte in the target can be determined. The target can be any target containing at least one analyte of interest that one may wish to detect and which indicates an actual or possible abnormal or normal condition, such as an abnormal medical pathology. The target can be a human or animal. In another embodiment, an analyte can be detected from a non-human or non-animal subject, for example a plant or tree, and the detected analyte can indicate an abnormal condition of the target, for example a disease in the case of a plant or tree. The analyte can be detected from a fluid, for example blood, interstitial fluid, cerebral spinal fluid, lymph fluid or urine; human tissue; animal tissue, plant tissue, an inanimate object, soil, genetic material, or a microbe.

The detection by the sensors described herein can be non-invasive meaning that the sensor remains outside the target, such as the human body, and the detection of the analyte occurs without requiring removal of fluid or other removal from the target, such as the human body. In the case of sensing in the human body, this non-invasive sensing may also be referred to as in vivo sensing. In other embodiments, the sensors described herein may be an in vitro sensor where the target containing the analyte has been removed from its host, for example from a human body.

The analyte(s) can be any analyte that one may wish to detect that may indicate an actual or possible abnormal or normal condition, such as an abnormal medical pathology. For example, in the case of a human target, the analyte(s) can include, but is not limited to, one or more of glucose, blood glucose, ketones, C-reactive proteins; blood alcohol, white blood cells, or luteinizing hormone. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

The sensor systems described herein operate by transmitting an electromagnetic signal (whether in the radio or microwave frequency range of the electromagnetic spectrum in FIGS. 1-5 and 6-9, and a harmonic thereof, toward and into a target using a transmit element such as a transmit antenna or a transmit LED. The transmission of the electromagnetic signal and its harmonic, according to at least some embodiments described herein, is simultaneous. A returning signal that results from both the transmission of the transmitted signal and its harmonic is detected by a receive element such as a receive antenna or a photodetector. The signal(s) detected by the receive element can be analyzed to detect the analyte based on the intensity of the received signal(s) and reductions in intensity at one or more frequencies where the analyte absorbs the transmitted signal. The signal detected by the receive element based on the intensity of the received signal in response to the simultaneous transmission of the harmonic signal may serve as a confirmation of the analysis or serve to call into question the accuracy of the analysis, prompting a re-test.

That is, the transmit circuit is configured to generate the electromagnetic transmit signal and a harmonic thereof to be transmitted respectively by at least two transmit elements. The receive circuit is electrically connectable to the at least one receive element, with the receive circuit being configured to receive the response detected by the at least one receive element in response to both the electromagnetic transmit signal and its simultaneously transmitted harmonic.

As referenced herein, a harmonic may refer to a signal or wave with a frequency that is a ratio of another reference wave or signal. Depending upon the integer multiple of the frequency to the original frequency, the respective harmonic wave may be implemented in increments of 2×, 3×, 4×, etc., of the reference wave.

FIGS. 1-5 illustrate a non-invasive analyte sensor system that uses multiple antennae including two or more transmit antennae and at least one receive antenna. The transmit antennae and the receive antenna can be located near the target and operated as further described herein to assist in detecting at least one analyte in the target. The transmit antennae each simultaneously transmit a signal at respective frequencies that are harmonics of each other, the two frequencies being in the radio or microwave frequency range, toward and into the target. The respective signals can be formed by separate signal portions, each having a discrete frequency that are harmonics of each other and that are transmitted simultaneously. In at least one embodiment, the signal from each of the respective transmit antennae may be part of a complex signal that includes a plurality of frequencies that are harmonics of the frequencies from the other one of the transmit antennae. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The one or more receive antenna detects a response resulting from transmission of the signal by each of the transmit antennae into the target containing the at least one analyte of interest.

The transmit antennae are respectively decoupled, i.e., detuned, from one another, and the transmit antennae are also respectively decoupled from the receive antenna. Decoupling refers to intentionally fabricating the configuration and/or arrangement of the transmit antennae and the receive antenna to minimize direct communication between the respective transmit antennae as well as between the respective transmit antennae and the receive antenna, preferably absent shielding. Shielding between the respective transmit antennae and between the respective transmit antennae and the receive antenna can be utilized. However, the transmit antennae and the receive antenna are decoupled even without the presence of shielding.

An example of detecting an analyte using a non-invasive spectroscopy sensor operating in the radio or microwave frequency range of the electromagnetic spectrum is described in WO 2019/217461, the entire contents of which are incorporated herein by reference. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e. without demultiplexing the complex signal) to detect the analyte as long as the detected signal provides enough information to make the analyte detection. In addition, the signal(s) detected by the receive antenna can be separate signal portions, each having a discrete frequency.

Referring now to FIG. 1, an embodiment of a non-invasive analyte sensor system with a non-invasive analyte sensor 5 is illustrated. The sensor 5 is depicted relative to a target 7 (in this example in the form of a human or animal or, even more particularly, a cell or tissue thereof) that contains an analyte of interest 9. In this example, the sensor 5 is depicted as including an antenna array that includes a transmit antennas array 11 (hereinafter “transmit antennae 11”) and a receive antenna/element 13 (hereinafter “receive antenna 13”). The sensor 5 further includes a transmit circuit 15, a receive circuit 17, and a controller 19. As discussed further below, the sensor 5 can also include a power supply, such as a battery (not shown in FIG. 1). In some embodiments, power can be provided from mains power, for example by plugging the sensor 5 into a wall socket via a cord connected to the sensor 5. The sensor 5 may be configured as a wearable device that is configured to be worn around the wrist (see FIG. 6), configured as a table top device (FIG. 7), used in an in vitro detector (see FIG. 8), or used in a non-human/animal version for example detection in an industrial process such as in a flowing fluid (see FIG. 9).

The transmit antennae 11 are each positioned, arranged and configured to transmit a respective signal 21 that is in the radio frequency (RF) or microwave range of the electromagnetic spectrum into the target 7 by one of the antennae 11, as well as a harmonic thereof by another of the antennae 11. The transmit antennae 11 can each be an electrode or any other suitable transmitter of electromagnetic signals in the radio frequency (RF) or microwave range. The transmit antennae 11 can each have any arrangement and orientation relative to the target 7 that is sufficient to allow the analyte sensing to take place. In one non-limiting embodiment, the transmit antennae 11 can each be arranged to face in a direction that is substantially toward the target 7.

The signal 21 transmitted by respective ones of the transmit antennae 11 is generated by the transmit circuit 15 which is electrically connectable to each of the transmit antennae 11. The transmit circuit 15 can have any configuration that is suitable to generate a transmit signal to be transmitted by the respective ones of transmit antennae 11. Transmit circuits for generating transmit signals in the RF or microwave frequency range, as well as a harmonic thereof, are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 can have at least two discrete frequencies (i.e. a plurality of discrete frequencies), each of which is in the range from about 10 kHz to about 100 GHz, as well as harmonics of one or more of the generated frequencies. In another embodiment, each of the at least two discrete frequencies, as well as the harmonics thereof, can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 300 MHz to about 6000 MHz. In an embodiment, the transmit circuit 15 can be configured to produce a complex transmit signal, the complex signal including a plurality of signal components, each of the signal components having a different frequency; and also produce another complex transmit signal that includes a plurality of signal components, each having a harmonic of the different frequencies in the other complex transmit signal. The complex signals can be generated, respectively, by blending or multiplexing multiple signals together followed by transmitting the complex signal whereby the plurality of frequencies, and their respective harmonics, are transmitted at the same time by respective ones of antennae 11.

The receive antenna 13 is positioned, arranged, and configured to detect one or more electromagnetic response signals 23 that result from the transmission of the transmit signal 21 by the respective transmit antennae 11 into the target 7 and impinging on the analyte 9, and also detect one or more electromagnetic response signals 23 that result from the transmission of a harmonic of the transmit signal 21 by another of the transmit antennae 11 into the target 7 and impinging on the analyte 9. The receive antenna 13 can be an electrode or any other suitable receiver of electromagnetic signals in the radio frequency (RF) or microwave range. In an embodiment, the receive antenna 13 is configured to detect electromagnetic signals having at least two frequencies, as well as harmonics thereof, each of which is in the range from about 10 kHz to about 100 GHz, or in another embodiment a range from about 300 MHz to about 6000 MHz, and the harmonics thereof. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19. In an embodiment, at least one of the receive circuit 17 or the controller 19 can be configured to decompose or demultiplex a complex signal, detected by the receive antenna 13, including a plurality of signal components each at different frequencies into each of the constituent signal components. In an embodiment, decomposing the complex signal can include applying a Fourier transform to the detected complex signals. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection.

The controller 19 controls the operation of the sensor 5. The controller 19, for example, can direct the transmit circuit 15 to generate a transmit signal to be transmitted by one of the transmit antennae 11, as well as a harmonic thereof to be simultaneously transmitted by another one of the transmit antennae 11. The controller 19 further receives signals corresponding to the transmit signal and its harmonic from the receive circuit 17. The controller 19 can optionally process the signals from the receive circuit 17 to detect the analyte(s) 9 in the target 7. In one embodiment, the controller 19 may verify or affirm the detection of the analyte(s) 9 in target 7 based on processing of signals from the receive circuit 17 that are in response to simultaneous transmission of a transmit signal and another signal with the harmonic thereof. Further, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the analyte(s) 9, confirm the detection of the analyte(s) 9 based on signals in response to simultaneous transmission of a transmit signal and another transmit signal that is a harmonic of the other transmit signals, and develop the analyte database. If provided, the external device 25 may be used to provide communication between the sensor 5 and the remote server 27, for example using a wired data connection or via a wireless data connection or Wi-Fi of the external device 25 to provide the connection to the remote server 27.

With continued reference to FIG. 1, the sensor 5 may include a sensor housing 29 (shown in dashed lines) that defines an interior space 31. Components of the sensor 5 may be attached to and/or disposed within the housing 29. For example, the transmit antennae 11 and the receive antenna 13 are attached to the housing 29. In some embodiments, the antennae 11, as well as antenna 13, may be entirely or partially within the interior space 31 of the housing 29. In some embodiments, the antennae 11, as well as antennae 13, may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

The receive antenna 13 is decoupled or detuned with respect to the transmit antennae 11 such that electromagnetic coupling between the transmit antennae 11 and the receive antenna 13 is reduced. The decoupling of the transmit antennae 11 and the receive antenna 13 increases the portion of the signal(s) detected by the receive antenna 13 that is the response signal(s) 23 from the target 7, and minimizes direct receipt of the transmitted signal 21 by the receive antenna 13. The decoupling of the transmit antennae 11 and the receive antenna 13 results in transmission from the transmit antennae 11 to the receive antenna 13 having a reduced forward gain (S₂₁) and an increased reflection at output (S₂₂) compared to antenna systems having coupled transmit and receive antennas.

In an embodiment, coupling between the respective transmit antennae 11 and the receive antenna 13 is 95% or less. In another embodiment, coupling between the respective transmit antennae 11 and the receive antenna 13 is 90% or less. In another embodiment, coupling between the respective transmit antennae 11 and the receive antenna 13 is 85% or less. In another embodiment, coupling between the respective transmit antennae 11 and the receive antenna 13 is 75% or less.

Any technique for reducing coupling between the respective transmit antennae 11 and the receive antenna 13 can be used. For example, the decoupling between the respective transmit antennae 11 and the receive antenna 13 can be achieved by one or more intentionally fabricated configurations and/or arrangements between the respective transmit antennae 11 and the receive antenna 13 that is sufficient to decouple the respective transmit antennae 11 and the receive antenna 13 from one another.

For example, in one embodiment described further below, the decoupling of the respective transmit antennae 11 and the receive antenna 13 can be achieved by intentionally configuring the respective transmit antennae 11 and the receive antenna 13 to have different geometries from one another. Intentionally different geometries refer to different geometric configurations of the transmit antennae 11 and receive antenna 13 that are intentional. Intentional differences in geometry are distinct from differences in geometry of transmit and receive antennas that may occur by accident or unintentionally, for example due to manufacturing errors or tolerances.

Another technique to achieve decoupling of the respective transmit antennae 11 and the receive antenna 13 is to provide appropriate spacing between each of antennae 11, 13 that is sufficient to decouple the respective antennae 11, 13 and force a proportion of the electromagnetic lines of force of the transmitted signal 21 into the target 7 thereby minimizing or eliminating as much as possible direct receipt of electromagnetic energy by the receive antenna 13 directly from the respective transmit antennae 11 without traveling into the target 7. The appropriate spacing between each of the antennae 11, 13 can be determined based upon factors that include, but are not limited to, the output power of the signal from the respective transmit antennae 11, the size of the respective antennae 11, 13, the frequency or frequencies of the transmitted signal, including harmonic thereof, and the presence of any shielding between the antennas. This technique helps to ensure that the response detected by the receive antenna 13 is measuring the analyte(s) 9 and is not just the transmitted signal 21 flowing directly from the transmit antenna 11 to the receive antenna 13. In some embodiments, the appropriate spacing between the respective antennae 11, 13 can be used together with the intentional difference in geometries of the respective antennae 11, 13 to achieve decoupling.

In one embodiment, the transmit signals that are transmitted by the respective transmit antennae 11 can have at least two different frequencies, as well as respective harmonics thereof, for example upwards of 7 to 12 different and discrete frequencies. In another embodiment, the transmit signal can be a series of discrete, separate signals with each separate signal having a single frequency or multiple different frequencies.

In one embodiment, the transmit signal (or each of the transmit signals) and harmonic thereof can be transmitted simultaneously over a transmit time that is less than, equal to, or greater than about 300 ms. In another embodiment, the transmit time can be less than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal, and one or more harmonics thereof, can be transmitted multiple times, simultaneously, and then the transmit time can be averaged. In another embodiment, the transmit signal (or each of the transmit signals) can be transmitted with a duty cycle that is less than or equal to about 50%.

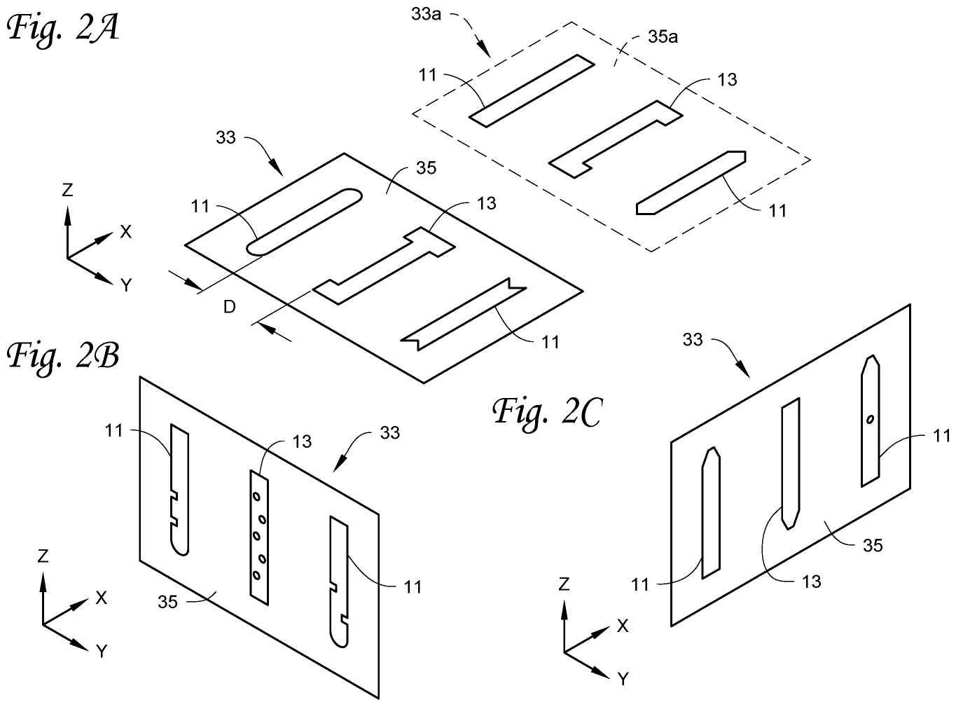

FIGS. 2A-2C illustrate examples of antenna arrays 33 that can be used in the sensor system 5 and how the antenna arrays 33 can be oriented. Many orientations of the antenna arrays 33 are possible, and any orientation can be used as long as the sensor 5 can perform its primary function of sensing the analyte(s) 9.

In FIG. 2A, the antenna array 33 includes the transmit antennae 11 and the receive antenna 13 disposed on a substrate 35 which may be substantially planar. This example depicts the array 33 disposed substantially in an X-Y plane. In this example, dimensions of the antennae 11, 13 in the X and Y-axis directions can be considered lateral dimensions, while a dimension of the antennae 11, 13 in the Z-axis direction can be considered a thickness dimension. In this example, each of the antennae 11, 13 has at least one lateral dimension (measured in the X-axis direction and/or in the Y-axis direction) that is greater than the thickness dimension thereof (in the Z-axis direction). In other words, the respective transmit antennae 11 and the receive antenna 13 are each relatively flat or of relatively small thickness in the Z-axis direction compared to at least one other lateral dimension measured in the X-axis direction and/or in the Y-axis direction.

In use of the embodiment in FIG. 2A, the sensor and the array 33 may be positioned relative to the target 7 such that the target 7 is below the array 33 in the Z-axis direction or above the array 33 in the Z-axis direction whereby one of the faces of the antennas 11, 13 face toward the target 7. Alternatively, the target 7 can be positioned to the left or right sides of the array 33 in the X-axis direction whereby one of the ends of each one of the antennae 11, 13 face toward the target 7. Alternatively, the target 7 can be positioned to the sides of the array 33 in the Y-axis direction whereby one of the sides of each one of the respective antennae 11, 13 face toward the target 7.

The sensor 5 can also be provided with one or more additional antenna arrays in addition the antenna array 33. For example, FIG. 2A also depicts an optional second antenna array 33a that includes the transmit antennae 11 and the receive antenna 13 disposed on a substrate 35a which may be substantially planar. Like the array 33, the array 33a may also be disposed substantially in the X-Y plane, with the arrays 33, 33a spaced from one another in the X-axis direction.

In FIG. 2B, the antenna array 33 is depicted as being disposed substantially in the Y-Z plane. In this example, dimensions of the respective antennae 11, 13 in the Y and Z-axis directions can be considered lateral dimensions, while a dimension of the respective antennae 11, 13 in the X-axis direction can be considered a thickness dimension. In this example, each of the antennas 11, 13 has at least one lateral dimension (measured in the Y-axis direction and/or in the Z-axis direction) that is greater than the thickness dimension thereof (in the X-axis direction). In other words, the transmit antennae 11 and the receive antenna 13 are each relatively flat or of relatively small thickness in the X-axis direction compared to at least one other lateral dimension measured in the Y-axis direction and/or in the Z-axis direction.

In use of the embodiment in FIG. 2B, the sensor and the array 33 may be positioned relative to the target 7 such that the target 7 is below the array 33 in the Z-axis direction or above the array 33 in the Z-axis direction whereby one of the ends of each one of the antennas 11, 13 face toward the target 7. Alternatively, the target 7 can be positioned in front of or behind the array 33 in the X-axis direction whereby one of the faces of each one of the respective antennae 11, 13 face toward the target 7. Alternatively, the target 7 can be positioned to one of the sides of the array 33 in the Y-axis direction whereby one of the sides of each one of the respective antennae 11, 13 face toward the target 7.

In FIG. 2C, the antenna array 33 is depicted as being disposed substantially in the X-Z plane. In this example, dimensions of the respective antennae 11, 13 in the X and Z-axis directions can be considered lateral dimensions, while a dimension of the antennas 11, 13 in the Y-axis direction can be considered a thickness dimension. In this example, each of the respective antennae 11, 13 has at least one lateral dimension (measured in the X-axis direction and/or in the Z-axis direction) that is greater than the thickness dimension thereof (in the Y-axis direction). In other words, the respective transmit antennae 11 and the receive antenna 13 are each relatively flat or of relatively small thickness in the Y-axis direction compared to at least one other lateral dimension measured in the X-axis direction and/or in the Z-axis direction.

In use of the embodiment in FIG. 2C, the sensor and the array 33 may be positioned relative to the target 7 such that the target 7 is below the array 33 in the Z-axis direction or above the array 33 in the Z-axis direction whereby one of the ends of each one of the respective antennae 11, 13 face toward the target 7. Alternatively, the target 7 can be positioned to the left or right sides of the array 33 in the X-axis direction whereby one of the sides of each one of the respective antennae 11, 13 face toward the target 7. Alternatively, the target 7 can be positioned in front of or in back of the array 33 in the Y-axis direction whereby one of the faces of each one of the respective antennae 11, 13 face toward the target 7.

The arrays 33, 33a in FIGS. 2A-2C need not be oriented entirely within a plane such as the X-Y plane, the Y-Z plane or the X-Z plane. Instead, the arrays 33, 33a can be disposed at angles to the X-Y plane, the Y-Z plane and the X-Z plane.

Decoupling Antennas Using Differences in Antenna Geometries

As mentioned above, one technique for decoupling the respective transmit antennae 11 from the receive antenna 13 is to intentionally configure the transmit antenna 11 and the receive antenna 13 to have intentionally different geometries. Intentionally different geometries refers to differences in geometric configurations of the respective transmit antennae 11 and receive antenna 13 that are intentional, and is distinct from differences in geometry of the respective transmit antennae 11 and receive antenna 13 that may occur by accident or unintentionally, for example due to manufacturing errors or tolerances when fabricating the respective antennae 11, 13.

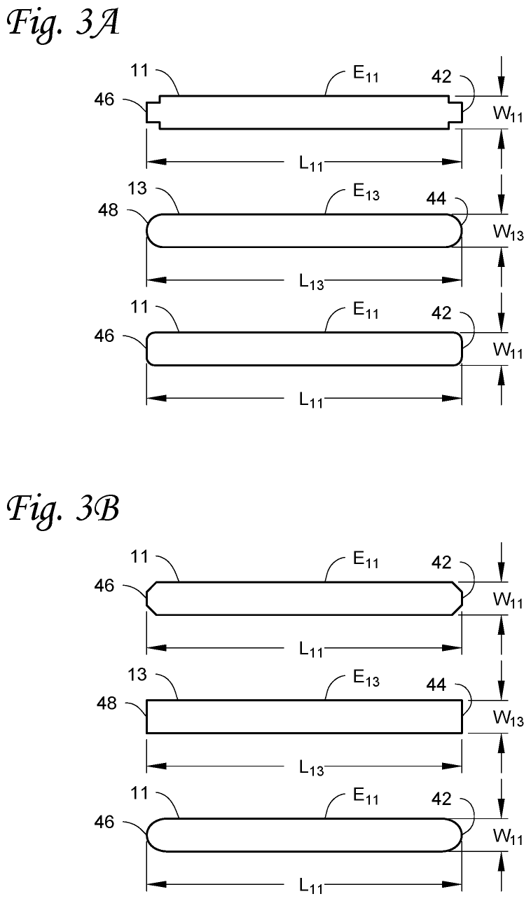

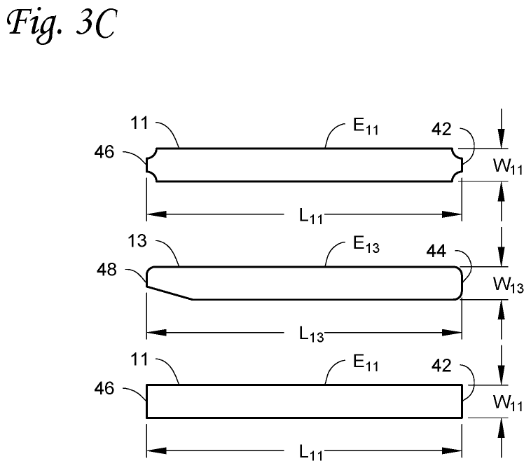

The different geometries of the respective antennae 11, 13 may manifest itself, and may be described, in a number of different ways. For example, in a plan view of each of the respective antennae 11, 13 (such as in FIGS. 3A-C), the shapes of the perimeter edges of the respective antennae 11, 13 may be different from one another. The different geometries may result in the respective antennae 11, 13 having different surface areas in plan view. The different geometries may result in the respective antennae 11, 13 having different aspect ratios in plan view (i.e. a ratio of their sizes in different dimensions; for example, as discussed in further detail below, the ratio of the length divided by the width of the respective antennae 11 may be different than the ratio of the length divided by the width for the antenna 13). In some embodiments, the different geometries may result in the respective antennae 11, 13 having any combination of different perimeter edge shapes in plan view, different surface areas in plan view, and/or different aspect ratios. In some embodiments, the respective antennae 11, 13 may have one or more holes formed therein (see FIG. 2B) within the perimeter edge boundary, or one or more notches formed in the perimeter edge (see FIG. 2B).

So as used herein, a difference in geometry or a difference in geometrical shape of the respective antennae 11, 13 refers to any intentional difference in the figure, length, width, size, shape, area closed by a boundary (i.e. the perimeter edge), etc. when the respective antennae 11, 13 is viewed in a plan view.

The respective antennae 11, 13 can have any configuration and can be formed from any suitable material that allows them to perform the functions of the respective antennae 11, 13 as described herein. In one embodiment, the respective antennae 11, 13 can be formed by strips of material. A strip of material can include a configuration where the strip has at least one lateral dimension thereof greater than a thickness dimension thereof when the antenna is viewed in a plan view (in other words, the strip is relatively flat or of relatively small thickness compared to at least one other lateral dimension, such as length or width when the antenna is viewed in a plan view as in FIGS. 3A-C). A strip of material can include a wire. The respective antennae 11, 13 can be formed from any suitable conductive material(s) including metals and conductive non-metallic materials. Examples of metals that can be used include, but are not limited to, copper or gold. Another example of a material that can be used is non-metallic materials that are doped with metallic material to make the non-metallic material conductive.

In FIGS. 2A-2C, the respective antennae 11, 13 within each one of the arrays 33, 33a have different geometries from one another. In addition, FIGS. 3A-C illustrate plan views of additional examples of the respective antennae 11, 13 having different geometries from one another. The examples in FIGS. 2A-2C and 3A-C are not exhaustive and many different configurations are possible.

FIG. 3A illustrates a plan view of an antenna array having two antennas with different geometries. In this example, the respective antennae 11, 13 are illustrated as substantially linear strips each with a lateral length L₁₁, L₁₃, a lateral width W₁₁, W₁₃, and a perimeter edge E₁₁, E₁₃. The perimeter edges E₁₁, E₁₃ extend around the entire periphery of the respective antennae 11, 13 and bound an area in plan view. In this example, the lateral length L₁₁, L₁₃ and/or the lateral width W₁₁, W₁₃ is greater than a thickness dimension of the respective antennae 11, 13 extending into/from the page when viewing FIG. 3A. In this example, the respective antennae 11, 13 differ in geometry from one another in that the shapes of the ends of the respective antennae 11, 13 differ from one another. For example, when viewing FIG. 3A, the right end 42 of the respective antennae 11 has a different shape than the right end 44 of the antenna 13. Similarly, the left end 46 of the respective antennae 11 may have a similar shape as the right end 42, but differs from the left end 48 of the antenna 13 which may have a similar shape as the right end 44. It is also possible that the lateral lengths L₁₁, L₁₃ and/or the lateral widths W₁₁, W₁₃ of the respective antennae 11, 13 could differ from one another.

FIG. 3B illustrates another plan view of an antenna array having two antennas with different geometries that is somewhat similar to FIG. 3A. In this example, the respective antennae 11, 13 are illustrated as substantially linear strips each with the lateral length L₁₁, L₁₃, the lateral width W₁₁, W₁₃, and the perimeter edge E₁₁, E₁₃. The perimeter edges E₁₁, E₁₃ extend around the entire periphery of the respective antennae 11, 13 and bound an area in plan view. In this example, the lateral length L₁₁, L₁₃ and/or the lateral width W₁₁, W₁₃ is greater than a thickness dimension of the respective antennae 11, 13 extending into/from the page when viewing FIG. 3B. In this example, the respective antennae 11, 13 differ in geometry from one another in that the shapes of the ends of the respective antennae 11, 13 differ from one another. For example, when viewing FIG. 3B, the right end 42 of the respective antennae 11 has a different shape than the right end 44 of the antenna 13. Similarly, the left end 46 of the respective antennae 11 may have a similar shape as the right end 42, but differs from the left end 48 of the antenna 13 which may have a similar shape as the right end 44. In addition, the lateral widths W₁₁, W₁₃ of the respective antennae 11, 13 differ from one another. It is also possible that the lateral lengths L₁₁, L₁₃ of the respective antennae 11, 13 could differ from one another.

FIG. 3C illustrates another plan view of an antenna array having two antennas with different geometries that is somewhat similar to FIGS. 3A and 3B. In this example, the respective antennae 11, 13 are illustrated as substantially linear strips each with the lateral length L₁₁, L₁₃, the lateral width W₁₁, W₁₃, and the perimeter edge E₁₁, E₁₃. The perimeter edges E₁₁, E₁₃ extend around the entire periphery of the respective antennae 11, 13 and bound an area in plan view. In this example, the lateral length L₁₁, L₁₃ and/or the lateral width W₁₁, W₁₃ is greater than a thickness dimension of the respective antennae 11, 13 extending into/from the page when viewing FIG. 3C. In this example, the antennas 11, 13 differ in geometry from one another in that the shapes of the ends of the respective antennae 11, 13 differ from one another. For example, when viewing FIG. 3C, the right end 42 of the respective antennae 11 has a different shape than the right end 44 of the antenna 13. Similarly, the left end 46 of the respective antennae 11 may have a similar shape as the right end 42, but differs from the left end 48 of the antenna 13 which may have a similar shape as the right end 44. In addition, the lateral widths W₁₁, W₁₃ of the respective antennae 11, 13 differ from one another. It is also possible that the lateral lengths L₁₁, L₁₃ of the respective antennae 11, 13 could differ from one another.

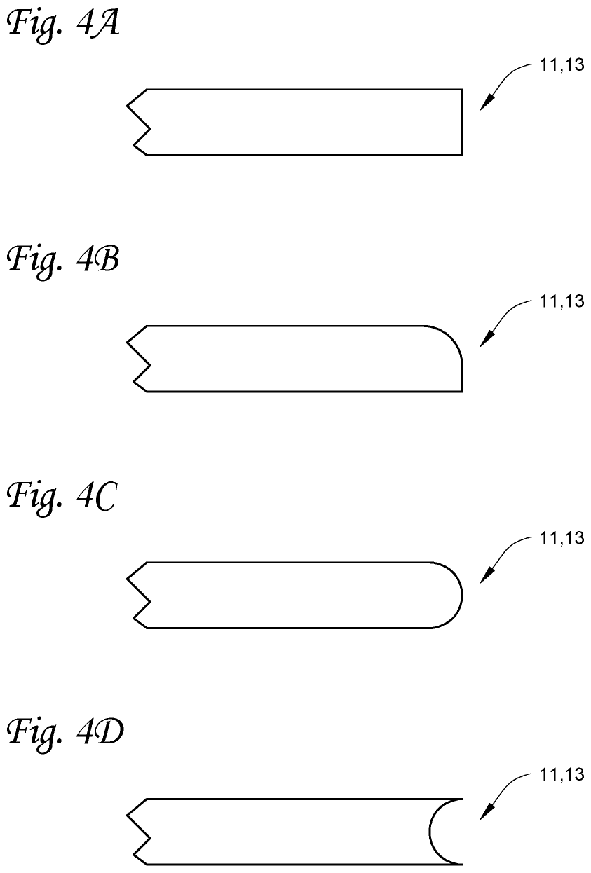

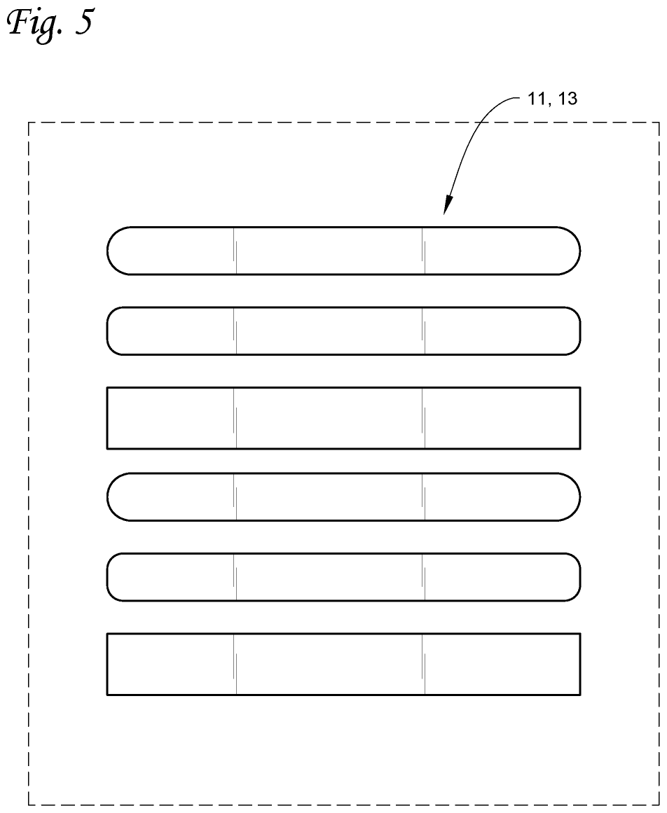

FIGS. 4A-D are plan views of additional examples of different shapes that the ends of the transmit and receive respective antennae 11, 13 can have to achieve differences in geometry. Either one of, or both of, the ends of the antennas 11, 13 can have the shapes in FIGS. 4A-D, including in the embodiments in FIGS. 3A-C. FIG. 4A depicts the end as being generally rectangular. FIG. 4B depicts the end as having one rounded corner while the other corner remains a right angle. FIG. 4C depicts the entire end as being rounded or outwardly convex. FIG. 4D depicts the end as being inwardly concave. Many other shapes are possible.

FIG. 5 illustrates another plan view of an antenna array having six antennas illustrated as substantially linear strips. In this example, the antennas differ in geometry from one another in that the shapes of the ends of the antennas, the lateral lengths and/or the lateral widths of the antennas differ from one another.

Another technique to achieve decoupling of the antennas is to use an appropriate spacing between each antenna with the spacing being sufficient to force most or all of the signal(s) transmitted by the transmit antenna into the target, thereby minimizing the direct receipt of electromagnetic energy by the receive antenna directly from the transmit antenna. The appropriate spacing can be used by itself to achieve decoupling of the antennas. In another embodiment, the appropriate spacing can be used together with differences in geometry of the antennas to achieve decoupling.

Referring to FIG. 2A, there is a spacing D between the respective transmit antennae 11 and the receive antenna 13 at the location indicated. The spacing D between the respective antennae 11, 13 may be constant over the entire length (for example in the X-axis direction) of each respective antennae 11, 13, or the spacing D between the respective antennae 11, 13 could vary. Any spacing D can be used as long as the spacing D is sufficient to result in most or all of the signal(s) transmitted by the respective transmit antennae 11 reaching the target and minimizing the direct receipt of electromagnetic energy by the receive antenna 13 directly from the respective transmit antennae 11, thereby decoupling the respective antennae 11, 13 from one another.

In addition, there is preferably a maximum spacing and a minimum spacing between the respective transmit antennae 11 and the receive antenna 13. The maximum spacing may be dictated by the maximum size of the housing 29. In one embodiment, the maximum spacing can be about 50 mm. In one embodiment, the minimum spacing can be from about 1.0 mm to about 5.0 mm.

FIG. 6 illustrates an example use of the sensor 5 of FIG. 1 in the form of a body wearable sensor, in particular a watch-like device 90 worn around the wrist. The sensor 5 is incorporated into a sensor body 92 that is fastened to the wrist by a strap 94 that extends around the wrist.

FIG. 7 illustrates an example use of the sensor 5 of FIG. 1 in the form of a tabletop device 100. The term “tabletop” is used interchangeably with “countertop” and refers to a device that is intended to reside on a top surface of a structure such as, but not limited to, a table, counter, shelf, another device, or the like during use. In some embodiments, the device 100 can be mounted on a vertical wall. The device 100 is configured to obtain a real-time, on-demand reading of an analyte in a user such as, but not limited to, obtaining a glucose level reading of the user using the non-invasive analyte sensor 5 incorporated into the device 100. The device 100 is illustrated as being generally rectangular box shaped. However, the device 100 can have other shapes such as cylindrical, square box, triangular and many other shapes. The device 100 includes a housing 102, a reading area 104, for example on a top surface of the housing 102, where the respective antennae 11, 13 of the sensor 5 are positioned to be able to obtain a reading, and a display screen 106, for example on the top surface of the housing 102, for displaying data such as results of a reading by the sensor 5. Power for the device 100 can be provided via a power cord 108 that plugs into a wall socket. The device 100 may also include one or more batteries which act as a primary power source for the device 100 instead of power provided via the power cord 108 or the one or more batteries can act as a back-up power source in the event power is not available via the power cord 108. A reading by the device 100 can be triggered with a trigger button 110. An on/off power button or switch 112 can be provided anywhere on the device 100 to power the device 100 on and off. The on/off power button or switch 112 could also function as the trigger button instead of the trigger button 110. Alternatively, the trigger button 110 may act as an on/off power button to power the device 100 on and off, as well as trigger a reading.

FIG. 8 illustrates the sensor 5 of FIG. 1 incorporated into an in vitro sensor 120 that is configured to operate with an in vitro sample that is held in a sample container 122 that contains a sample to be analyzed, where the container 122 is held in a sample chamber 124. The sensor 120 can include additional features that are similar to the features of the housing disclosed in U.S. Pat. No. 9,041,920 the entire contents of which are incorporated herein by reference.

FIG. 9 illustrates the sensor 5 of FIG. 1 as an in vitro sensor 130 in an industrial process, for example with an in vitro fluid passageway 132 through which an in vitro fluid flows as indicated by the arrow A. The sensor 130 can be positioned outside the passageway 132 as illustrated, or the sensor 130 can be positioned within the passageway 132. The sensor 130 can be used in any application that can transmit the signal(s) into a target and receive a response.

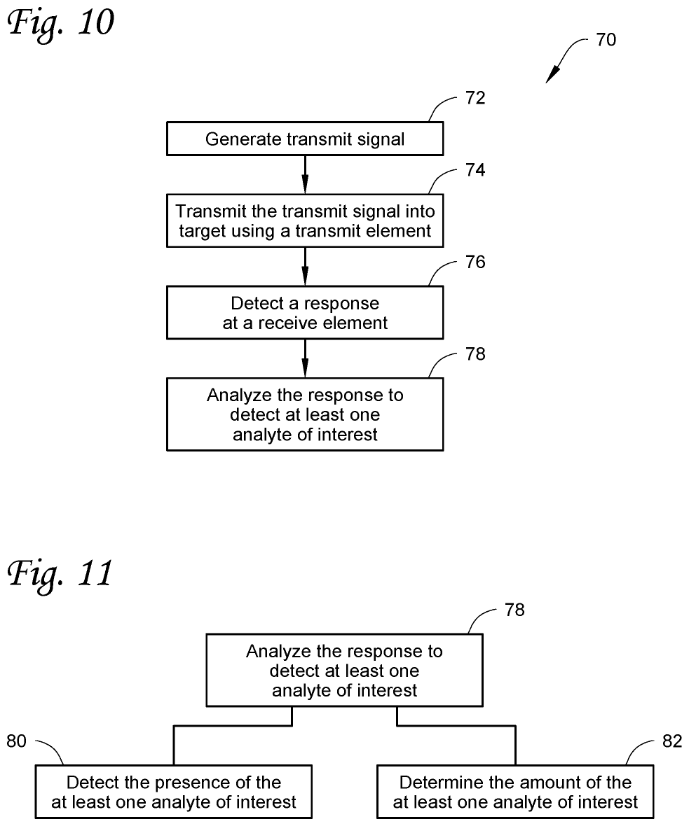

With reference now to FIG. 10, one embodiment of a method 70 for detecting at least one analyte in a target is depicted. The method in FIG. 10 can be practiced using any of the embodiments of sensor devices described herein including the sensor 5 and the sensor 50. In order to detect the analyte, the sensor 5, 50 is placed in relatively close proximity to the target. Relatively close proximity means that the sensor 5, 50 can be close to but not in direct physical contact with the target, or alternatively the sensor 5, 50 can be placed in direct, intimate physical contact with the target. The spacing (if any) between the sensor 5, 50 and the target can be dependent upon a number of factors, such as the power of the transmitted signal. Assuming the sensor 5, 50 is properly positioned relative to the target, at box 72 the transmit signals are generated, for example by the transmit circuit 15. The transmit signals are then provided to the transmit element (11 or 54) which, at box 74, transmits the transmit signals toward and into the target. The transmit signals, according to at least one embodiment, are harmonics of one another. At box 76, a response resulting from the transmit signals contacting the analyte(s) is then detected by the receive element (13, 54, or 56). The receive circuit obtains the detected response from the receive element and provides the detected response to the controller. At box 78, the detected response can then be analyzed to detect at least one analyte. The analysis can be performed by the controller 19 and/or by the external device 25 and/or by the remote server 27.

Referring to FIG. 11, the analysis at box 78 in the method 70 can take a number of forms. In one embodiment, at box 80, the analysis can simply detect the presence of the analyte, i.e. is the analyte present in the target. Alternatively, at box 82, the analysis can determine the amount of the analyte that is present.

For example, in the case of the sensor being the sensor 5 and the signal being in the radio frequency range, the interaction between the transmitted signal and the analyte may, in some cases, increase the intensity of the signal(s) that is detected by the receive antenna, and may, in other cases, decrease the intensity of the signal(s) that is detected by the receive antenna. For example, in one non-limiting embodiment, when analyzing the detected response, compounds in the target, including the analyte of interest that is being detected, can absorb some of the transmit signal, with the absorption varying based on the frequency of the transmit signal. The response signal detected by the receive antenna may include drops in intensity at frequencies where compounds in the target, such as the analyte, absorb the transmit signal. The frequencies of absorption are particular to different analytes. The response signal(s) detected by the receive antenna can be analyzed at frequencies that are associated with the analyte of interest to detect the analyte based on drops in the signal intensity corresponding to absorption by the analyte based on whether such drops in signal intensity are observed at frequencies that correspond to the absorption by the analyte of interest. A similar technique can be employed with respect to increases in the intensity of the signal(s) caused by the analyte.

Detection of the presence of the analyte, as well as confirmation or affirmation thereof, can be achieved, for example, by identifying a change in the signal intensity detected by the receive antenna at a known frequency associated with the analyte. The change may be a decrease in the signal intensity or an increase in the signal intensity depending upon how the transmit signal interacts with the analyte. The known frequency associated with the analyte can be established, for example, through testing of solutions known to contain the analyte. Determination of the amount of the analyte can be achieved, for example, by identifying a magnitude of the change in the signal at the known frequency, for example using a function where the input variable is the magnitude of the change in signal and the output variable is an amount of the analyte. The determination of the amount of the analyte can further be used to determine a concentration, for example based on a known mass or volume of the target. In an embodiment, presence of the analyte and determination of the amount of analyte may both be determined, for example by first identifying the change in the detected signal to detect the presence of the analyte, and then processing the detected signal(s) to identify the magnitude of the change to determine the amount.

In operation of either one of the sensors 5, 50 of FIGS. 1-9, one or more frequency sweeps or scan routines can implemented. The frequency sweeps can be implemented at a number of discrete frequencies (r frequency targets) over a range of frequencies.

In another embodiment, a non-invasive sensor can include aspects of both of the sensors 50. For example, a sensor can include both two or more antennae as described herein. The antennas can be used together to detect an analyte.

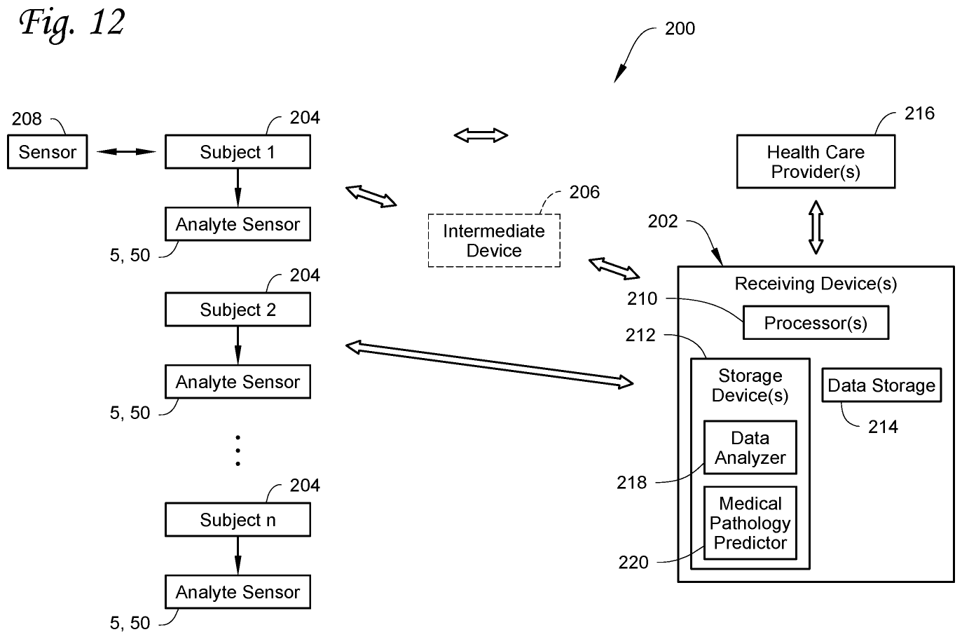

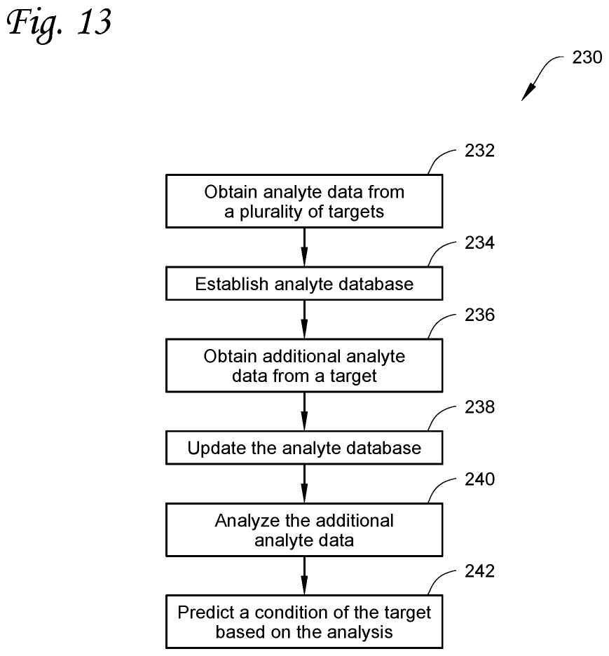

Referring now to FIGS. 12 and 13, systems and methods involving the use of the analyte sensors, for example similar to those described herein, to predict an actual or possible abnormal or normal condition, such as an abnormal medical condition, of a target are described. For sake of convenience, the systems and methods will be described as using the analyte sensors 5, 50 described herein with respect to FIGS. 1-9. In another embodiment, the systems and methods can use the analyte sensors disclosed in U.S. Pat. No. 10,548,503, U.S. Patent Application Publication 2019/0008422, or U.S. Patent Application Publication 2020/0187791, each of which is incorporated herein by reference in its entirety. Combinations of the features of the sensors 5, 50 described herein and disclosed in U.S. Pat. No. 10,548,503, U.S. Patent Application Publication 2019/0008422, or U.S. Patent Application Publication 2020/0187791 can be used.

Referring initially to FIG. 12, a predictive medical analytics system 200 according to one embodiment is illustrated. A similar system can be implemented with other targets. The system 200 includes a receiving device 202 that is configured to receive analyte data directly or indirectly from one or more of the analyte sensors 5, 50. Each sensor 5, 50 is interfaceable with a corresponding subject 204, for example a human or animal or cell or tissue thereof, for detecting at least one analyte in the subject 204. For example, the sensor 5, 50 may be worn by the subject 204, for example worn around the subjects wrist, or the sensor 5, 50 may be incorporated into a device, such as a table-top device or a hand-held device for detecting the analyte(s) in the subject 204. The sensor(s) 204 conducts a plurality of analyte sensing routines to sense at least one analyte in the subject 204, where the at least one analyte is an indicator of an abnormal medical pathology of the subject 204.

The analyte can be any analyte that is an indicator of an abnormal medical pathology due to the presence of the analyte and/or due to the concentration of the analyte. Many analytes as indicators of abnormal medical pathologies are possible, too numerous to mention. For example, the analyte can be glucose where glucose concentration levels (either high (i.e. hyperglycemia) or low (i.e. hypoglycemia)) over a period of time ae a well-known indicator of pre-diabetes or diabetes.

In another example, the analyte can be c-reactive proteins where high levels of c-reactive proteins are an indicator of diabetes, thrombotic events including myocardial infarction, and some cancers such as lung cancer and breast cancer. See Mankowski et al., “Association of C-Reactive Protein And Other Markers Of Inflammation With Risk Of Complications In Diabetic Subjects”, The Journal Of The International Federation Of Clinical Chemistry And Laboratory Medicine, March 2006; Allin et al., “Elevated C-reactive protein in the diagnosis, prognosis, and cause of cancer”, Crit Rev Clin Lab Sci, Jul-Aug 2011.

In another example, the analyte can be ketones where high levels of ketones are an indicator of hyperglycemia and diabetes. See Mahendran et al., Association of Ketone Body Levels With Hyperglycemia and Type 2 Diabetes in 9,398 Finnish Men“, Diabetes, Vol. 62, October 2013.

In another example, the analyte can be white blood cells where high levels of white blood cells are an indicator of alcoholic liver cirrhosis. See Alcoholic Liver Cirrhosis, https://www.healthline.com/health/alcoholic-liver-cirrhosis #symptoms, September 2018.

In another example, the analyte can be luteinizing hormone (LH) where too much or too little LH can be an indicator of abnormal medical pathology including infertility, menstrual difficulties in women, low sex drive in men, and early or delayed puberty in children. See Luteinizing Hormone (LH) Levels Test, https://medlineplus.gov/lab-tests/luteinizing-hormone-1h-levels-test/#:˜:text=This %20 test %20measures %20the %20level,helps %20control %20the %20menstrual %20cycle.

As shown in FIG. 12, the analyte sensor 5, 50 may be in wireless or wired communication with an intermediate device 206 which in turn is in wireless or wired communication with the receiving device 202, whereby the receiving device 202 indirectly receives the analyte data from the sensor 5, 50. The intermediate device 206 can be any device that can interface with the analyte sensor 5, 50 and the receiving device 202 including, but not limited to, a mobile device such as a mobile phone, a tablet computer, a laptop computer, or the like. The intermediate device 206 may also be a personal computer. The intermediate device 206 may also be a specially designed device that is created specifically to interface with the analyte sensor 5, 50 and the receiving device 202. The intermediate device 206 may be provided with an app designed by the entity that controls the receiving device 202 that allows the intermediate device 206 to function with the analyte sensor 5, 50 and the receiving device 202. The intermediate device 206 may be owned by the subject 204, or owned by a parent if the subject 204 is a child, or owned by a care giver if the subject 204 is under care of a care giver. Alternatively or additionally, the receiving device 202 may be in direct wired or wireless communication with the analyte sensor 204 whereby the receiving device 202 directly receives the analyte data from the sensor 5, 50.

As used herein, receiving analyte data includes receiving the analyte readings from the analyte sensor 5, 50 whereby the analyte sensor 5, 50 and/or the intermediate device 206 processes the signals detected by the receive element of the sensor 5, 50 during a scan routine to determine the presence and/or concentration of the analyte, with the processed analyte data (i.e. the analyte presence and/or concentration readings) being sent to the receiving device 202. Therefore, the detected signals may be processed entirely by the analyte sensor 5, 50, the detected signals may be entirely processed by the intermediate device 206, or the detected signals may be partially processed by the analyte sensor 5, 50 and partially by the intermediate device 206. Receiving analyte data as used herein also includes receiving raw analyte readings from the analyte sensor 5, and/or the intermediate device 206 whereby the raw signals detected by the receive element of the sensor 5, 50 are sent to the receive device 202 and the receive device 202 processes the raw signals to determine the presence and/or concentration of the analyte. Therefore, the detected signals may be processed entirely by the receiving device 202, or the receiving device 202 may finish processing the detected signals which have been partially processed by the analyte sensor 5, and/or the intermediate device 206. In another embodiment, the receive element 202 can receive both the processed analyte data and the raw analyte data, with the receive element 202 processing the raw data to determine the presence and/or concentration of the analyte for comparison to the received processed analyte data.

The analyte data is collected by the sensor 5, 50 over a period of time that is sufficient to indicate an actual or possible abnormal medical pathology or condition of the subject 204. The time period over which the analyte data is collected may vary based on a number of factors including, but not limited to, the subject 204, the analyte being detected, temporal factors (for example time of day, the day(s) of the week, month or year), and other factors. The time period over which the analyte data is collected can be measured in minutes, hours, days, months or even years. In one embodiment, the time period can be selected to minimize or avoid collecting analyte data encompassing natural or non-abnormal variations in the analyte of the subject 204 that may occur and that may not indicate an actual or possible abnormal medical pathology of the subject 204. In another embodiment, to err on the side of medical caution, the time period that is selected may include collecting analyte data that encompasses natural or normal variations in the analyte of the subject 204 that may occur whether or not all of the collected analyte data indicates an actual or possible abnormal medical pathology. For example, the plurality of analyte sensing routines can be conducted over a period of time of at least twenty four hours, 5 days, 1 week, 1 month, 3 months, 6 months, 9 months, 1 year, and may others. In still another embodiment, instead of collecting analyte data over a period of time, a single analyte reading can be used to predict an actual or possible abnormal medical pathology.

The scan routines conducted by the analyte sensor 5, 50 to obtain the analyte data can occur continuously over the time period, or at regular or irregular intervals over the time period. The scan routines can be conducted automatically under control of a control system. In another embodiment, the scan routines can be manually triggered by the subject 204. In still another embodiment, the scan routines can be conducted automatically with the subject 204 also able to trigger one or more manual scan routines upon demand.

The analyte data can be transmitted to and received by the receiving device 202 in multiple transmissions. For example, the analyte data collected by the analyte sensor 5, 50 can be transmitted to the receiving device 202 during or after each sensing routine over the sensing period. In another embodiment, the analyte data can be transmitted to and received by the receiving device 202 in a single transmission. For example, the sensor 5, 50 or the intermediate device 206 can store the analyte data from each scan routine and at the end of the sensing period, all of the analyte data from all of the scan routines can be transmitted to the receiving device 202.

In an embodiment, a second sensor 208 can be interfaceable with the subject 204 to detect second data of the subject 204 which is transmitted to the receiving device 202. The second sensor 208 can be a second analyte sensor 5, 50 that can detect the same or different analyte as the sensor 50, or the second sensor 208 can be a sensor that detects another variable of the subject 204 such as, but not limited to, heart rate, blood pressure, oxygen level, temperature, hydration, and others. The data from the second sensor 208 can be used together with the analyte data from the sensor 5, to predict the abnormal medical pathology of the subject 204.

The receiving device 202 includes one or more processors 210, one or more non-transitory machine/computer-readable storage mediums (i.e. storage device(s)) 212, and one or more data storage 214. The receiving device 202 may be a server or other computer hardware. The receiving device 202 may also be implemented in a cloud computing environment.

The processor(s) 210 can have any construction that is suitable for processing the analyte data received by the receiving device 202. The processor(s) 210 can be a microprocessor, microcontroller, embedded processor, a digital signal processor, or any other type of logic circuitry. The processor(s) 210 can be single core or multi-core.

The data storage 214 stores the analyte data received by the receiving device 202 and also stores the results of the data analysis performed by the receiving device 202. The data storage 214 may also store an analyte database that is established from analyte readings obtained from the subjects 204 over a period of time. The data storage 214 can be any form of long term data storage. The data storage 214 may be implemented by cloud storage, or by data storage at a single location.

The at least one storage device 212 comprises program instructions that are executable by the one or more processors 210 to configure the receiving device 202 to be able to receive the analyte data, to transmit data and/or commands to the analyte sensor 5, 50 and/or to the intermediate device 206, and optionally to communicate with one or more health care providers 216. The health care provider 216 can be the health care provider for the subject 204, for example a nurse, a doctor or other health care provider. The program instructions of the at least one storage device 212 can further control other functions of the receiving device 202 including general operation of the receiving device 202, including internal and external communications, and interactions between the various elements of the receiving device 202, and the like.

The at least one storage device 212 can further comprise program instructions that are executable by the one or more processors 210 to function as a data analyzer 218 that analyzes the analyte data received from the sensor 5, 50 and/or from the intermediate device 206. The data analyzer 218 functions to analyze the received analyte data to determine the presence of the analyte(s) and/or the concentration of the analyte(s) in the manner described above.

The at least one storage device 212 can further comprise program instructions that are executable by the one or more processors 210 to function as a medical pathology predictor 220 that uses the results of the analysis of the analyte data to predict an abnormal medical condition of the subject 204. For example, the medical pathology predictor 220 can use the analyte data to detect trends in the analyte suggesting an actual or possible abnormal medical condition. For example, the mere presence of an analyte can indicate a possible or actual abnormal medical condition. In another example, a detected analyte level over a certain threshold, or below a certain threshold, for a period of time can be suggestive of an actual or possible abnormal medical condition. In another example, significant changes in the analyte level can be suggestive of an actual or possible abnormal medical condition.

The receiving device 202 can generate an electronic report based on the results of the analysis of the received analyte data. The report can include the results of the analysis, including a positive or normal analysis (i.e. no abnormal medical pathology exists), or including a predicted abnormal medical pathology. In the case of a predicted abnormal medical pathology, the report may also include guidance to the subject 204 on how to rectify the abnormal medical pathology, or guidance to seek medical attention to confirm and address the abnormal medical pathology, or other guidance. The receiving device 202 may include a display that displays the report, or the receiving device 202 may transmit the electronic report to a location remote from the receiving device 202. For example, the electronic report may be transmitted to the intermediate device 206 and/or to the analyte sensor 5, 50 for display. The electronic report may be transmitted to the health care provider(s) 216 who in turn may provide the report to the subject 204 or otherwise report the results to the subject 204.

In one embodiment, all of the elements of the system 200, including the analyte sensor 5, the intermediate device 206 and the receiving device 202 may be provided from and controlled by a single entity. Or the entity may provide and control the analyte sensor 5, 50 and the receiving device 202, and provide an app for downloading by the subject 204 onto the intermediate device 206, for example a mobile phone or tablet owned by the subject 204, that configures the intermediate device to function with the analyte sensor 5, 50 and the intermediate device 202. Or the entity may provide and control the receiving device 202, and provide an app(s) for downloading by the subject 204 onto the intermediate device 206, for example a mobile phone or tablet owned by the subject 204 and for downloading onto the analyte sensor 5, 50, for example in the form of a smartwatch-like device owned by the subject 204, that configures the intermediate device 206 and the analyte sensor 5, 50 to function with the intermediate device 202.

A method 230 using the predictive medical analytics system 200 of FIG. 12 is illustrated in FIG. 13. The method 230 includes, at step 232, obtaining analyte data from a plurality of targets (such as the targets 204 in FIG. 12). The analyte data is obtained over a period of time, for example at least 24 hours, from each target as described herein using the analyte sensors described herein. For example, the analyte data can be sent to the receiving device 202 from the intermediate devices 206 which receive the analyte data from the analyte sensors 5, 50. The analyte data can be sent to the receiving device 202 in multiple transmissions or in a single transmission. In addition, the analyte data can be raw, unprocessed analyte data, or the analyte data can be processed data that has been processed by the intermediate device 206 and/or by the analyte sensor 5, 50.

At step 234, the analyte database is established based on the analyte data that has been obtained from the targets. The analyte data in the analyte database provides information on one or more analytes in the analyte data. For example, in the case of analyte data from human targets, the analyte data can indicate the presence and concentration of an analyte such as glucose as previously described herein. The use of analyte data from multiple targets over a prolonged period of time helps increase the confidence that the obtained data is accurate and reduces the impact of random variations in analyte levels in the targets.

Once the analyte database is established, at step 236 new or additional analyte data can be obtained from a target using one of the analyte sensors described herein. The new analyte data is obtained from the target over a period of time, for example 24 hours or more. The target can be one of the targets used to establish the analyte database, or the target can be a new target that is different from the targets used to establish the analyte database. According to at least some of the embodiments described and recited herein, the new or additional analyte data may be based on transmit signals that are harmonics of previously transmitted detect signals, the harmonics being simultaneously sent from a different one of antennae 11. In step 238, the new analyte data can optionally be added to the analyte database to update the analyte database.

In step 240, the new analyte data is analyzed based on the analyte database. For example, the new analyte data can be analyzed, for example using the medical pathology predictor 220 of FIG. 12, by comparing the new analyte data to the analyte data in the analyte database to determine the presence (or absence) of one or more analytes and/or determine a concentration of the one or more analytes using the analyte database. The analysis of the new analyte data, based on transmit signals that are harmonics of detect signals simultaneously sent from at least one separate transmit antenna, may affirm or confirm the presence (or absence) of the one or more analytes. At step 242, an actual or possible condition of the target can then be predicted based on the analysis of the new analyte data. For example, if the analysis reveals the presence of a particular analyte in the new analyte data, or reveals a particular concentration of a particular analyte, that can be an indicator of an abnormal (or normal) condition, such as an abnormal medical pathology of a human target.

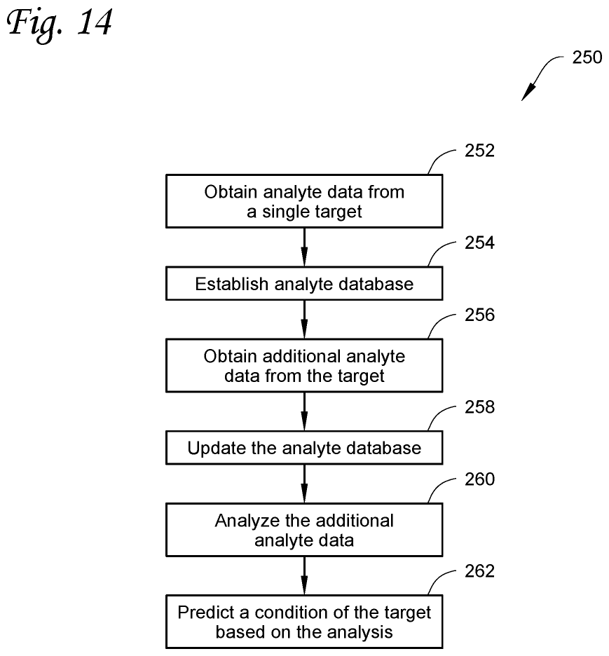

FIG. 14 illustrates another example of a method 250 of using the predictive medical analytics system 200 of FIG. 12. In this example, an analyte database that is specific to a single individual is established. The method 250 includes, at step 252, obtaining analyte data from a single target (such as one of the targets 204 in FIG. 12). The analyte data is obtained over a period of time, for example at least 24 hours, from the target as described herein using one or more of the analyte sensors described herein. For example, the analyte data can be sent to the receiving device 202 from the intermediate devices 206 which receive the analyte data from the analyte sensor 5, 50. The analyte data can be sent to the receiving device 202 in multiple transmissions or in a single transmission. In addition, the analyte data can be raw, unprocessed analyte data, or the analyte data can be processed data that has been processed by the intermediate device 206 and/or by the analyte sensor 5, 50.

At step 254, the analyte database is established based on the analyte data that has been obtained from the single target. The analyte data in the analyte database provides information on one or more analytes in the analyte data. For example, in the case of analyte data from a human target, the analyte data can indicate the presence and concentration of an analyte such as glucose as previously described herein. The use of analyte data from the single target over a prolonged period of time helps increase the confidence that the obtained data is accurate and reduces the impact of random variations in analyte levels in the target.

Once the analyte database is established, at step 256 new or additional analyte data can be obtained from the target using one of the analyte sensors described herein. The new analyte data is obtained from the target over a period of time, for example 24 hours or more. In step 258, the new analyte data can optionally be added to the analyte database to update the analyte database.

In step 260, the new analyte data is analyzed based on the analyte database. For example, the new analyte data can be analyzed, for example using the medical pathology predictor 220 of FIG. 12, by comparing the new analyte data to the analyte data in the analyte database to determine the presence (or absence) of one or more analytes and/or determine a concentration of the one or more analytes using the analyte database and/or determine a change in the analyte. At step 262, an actual or possible condition of the target can then be predicted based on the analysis of the new analyte data. For example, if the analysis reveals the presence of a particular analyte in the new analyte data, or reveals a particular concentration of a particular analyte, or reveals a significant change in analyte, that can be an indicator of an abnormal (or normal) condition, such as an abnormal medical pathology of a human target.

In FIGS. 13 and 14, any one or more of the establishment of the analyte databases, updating the analyte databases, analysis of the new analyte data, and predicting a condition of the target can be performed using artificial intelligence techniques, such as using machine learning techniques. For example, artificial intelligence software can be trained to recognize different signals, that are obtained by the analyte sensors described herein, that correspond to different analytes at different frequencies. The artificial intelligence software can also be trained to correlate the recognized signals and the corresponding analyte(s) to one or more corresponding determinations, such as an abnormal medical pathology associated with the corresponding analyte(s).

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

We claim:

1. A sensor system, comprising:
a detector array having at least two transmit elements and at least one receive element, the at least two transmit elements are positioned and arranged to respectively and simultaneously transmit a transmit signal and a harmonic of the transmit signal into a target, and the at least one receive element is positioned and arranged to detect responses resulting from transmission of both the transmit signal and the harmonic thereof by the at least two transmit elements into the target;
a transmit circuit that is electrically connectable to the at least two transmit elements, the transmit circuit is configured to generate the transmit signal and harmonic thereof to be transmitted by the at least two transmit elements, the transmit signal and the harmonic thereof are each in a radio or microwave frequency range of the electromagnetic spectrum;
a receive circuit that is electrically connectable to the at least one receive element, the receive circuit is configured to receive the responses detected by the at least one receive element.

2. The sensor system of claim 1, further comprising:
a control system connected to the transmit circuit and configured to implement at least first and second frequency sweeps by the at least two transmit elements, the first frequency sweep occurs over a first frequency range from a start frequency to an end frequency, the second frequency sweep occurs over a second frequency range from a start frequency to an end frequency, and the first frequency range and the second frequency range overlap one another;
the first frequency range and the second frequency range where they overlap have first frequency steps and second frequency steps, respectively; the first frequency steps are the same as the second frequency steps; each frequency step of the first frequency steps and the second frequency steps has an associated operation time, and the operation times of the first frequency steps are identical to the operation times of the second frequency steps.

3. The sensor system of claim 2, wherein the operation times of at least some of the first frequency steps are not equal to one another, and the operation times of at least some of the second frequency steps are not equal to one another.

4. The sensor system of claim 2, wherein the operation times of the first frequency steps are equal to one another, and the operation times of the second frequency steps are equal to one another.

5. The sensor system of claim 2, wherein each operation time includes a plurality of sub-operations.

6. The sensor system of claim 5, wherein the plurality of sub-operations of at least one of the operation times include a plurality of transmissions of signals having a respective frequency and their harmonics by the at least two transmit elements.

7. The sensor system of claim 1, wherein the detector array, the transmit circuit, and the receive circuit are incorporated into a wearable watch or a tabletop device.

8. The sensor system of claim 1, wherein the target is a body fluid, and the sensor system is configured to sense blood glucose, alcohol, white blood cells, or luteinizing hormone.

9. The sensor system of claim 1, wherein the at least two transmit elements and the at least one receive element comprise antennas, and the first frequency range and the second frequency range are each in a radio or microwave frequency range of the electromagnetic spectrum.

10. A method of operating a sensor system that includes a detector array having at least two transmit elements and at least one receive element, the at least two transmit elements are positioned and arranged to respectively transmit signals into a target, and the at least one receive element is positioned and arranged to detect responses resulting from transmission of the signals by the at least two transmit elements into the target, a transmit circuit that is electrically connectable to the at least two transmit elements where the transmit circuit is configured to generate the signals to be transmitted by the at least two transmit elements, and a receive circuit that is electrically connectable to the at least one receive element, the method comprising:
generating a transmit signal and a harmonic of the transmit signal using the transmit circuit, the transmit signal and the harmonic of the transmit signal are in a radio or microwave frequency range of the electromagnetic spectrum;
simultaneously transmitting the transmit signal and the harmonic of the transmit signal from the at least two transmit elements into the target;
detecting responses using the at least one receive element that result from the transmission of the transmit signal and the harmonic of the transmit signal into the target;
receiving the detected responses at the receive circuit.

11. The method of claim 10, further comprising:
controlling the sensor system to implement at least a first frequency sweep and a second frequency sweep by the at least two transmit elements, the first frequency sweep occurs over a first frequency range from a start frequency to an end frequency, the second frequency sweep occurs over a second frequency range from a start frequency to an end frequency, and the first frequency range and the second frequency range overlap one another, the first frequency range and the second frequency range where they overlap have first frequency steps and second frequency steps, respectively; the first frequency steps are the same as the second frequency steps; each frequency step of the first frequency steps and the second frequency steps has an associated operation time, and the operation times of the first frequency steps are identical to the operation times of the second frequency steps.

12. The method of claim 11, comprising controlling the sensor system so that the operation times of at least some of the first frequency steps are not equal to one another, and the operation times of at least some of the second frequency steps are not equal to one another.

13. The method of claim 11, comprising controlling the sensor system so that the operation times of the first frequency steps are equal to one another, and the operation times of the second frequency steps are equal to one another.

14. The method of claim 11, comprising controlling the sensor system so that each operation time includes a plurality of sub-operations.

15. The method of claim 14, comprising controlling the sensor system so that the plurality of sub-operations of at least one of the operation times include a plurality of transmissions of a signal having a frequency and a harmonic thereof by the at least two transmit elements.

16. The method of claim 10, wherein the detector array, the transmit circuit, and the receive circuit are incorporated into a wearable watch or a tabletop device.

17. The method of claim 10, wherein the target is a body fluid, and the sensor system is configured to sense blood glucose, blood alcohol, white blood cells, or luteinizing hormone.

18. The method of claim 11, wherein the at least two transmit elements and the at least one receive element comprise antennas, and the first frequency range and the second frequency range are each in a radio or microwave frequency range of the electromagnetic spectrum.
